(19) United States
(12) Patent Application Publication
(10) Pub. No.: US 2011/0040332 A1
(43) Pub. Date: Feb. 17, 2011

(54) SPINOUS PROCESS SPACER AND IMPLANTATION PROCEDURE

(75) Inventors: Brad S. Culbert, Rancho Santa Margarita, CA (US); Christopher Warren, Aliso Viejo, CA (US); Robert Flower, Sun City, CA (US); Fausto Olmos, Laguna Niguel, CA (US)

Correspondence Address: KNOBBE MARTENS OLSON & BEAR LLP, 2040 MAIN STREET, FOURTEENTH FLOOR, IRVINE, CA, 92614, US

(73) Assignee: INTERVENTIONAL SPINE, INC., Irvine, CA (US)

(21) Appl. No.: 12/854,774

(22) Filed: Aug. 11, 2010

Related U.S. Application Data
(60) Provisional application No. 61/233,097, filed on Aug. 11, 2009.

(51) Int. Cl. A61B 17/88

(52) U.S. Cl. 606/279

(57) ABSTRACT

A spinal fixation procedure and system are provided for fixing the spacing of an inferior vertebra relative to a superior vertebra. The procedure for implanting a spinous process spacer can comprise decorticating and/or forming a notch in adjacent spinous processes, measuring the distance between the notches formed in the spinous processes, and inserting an interspinous process implant such that the implant is fitted into the notches of the spinous processes. Other fixation devices, such as bone screws, can also be used for fixing the position of the vertebrae and to create facet fusion.

### CROSS-REFERENCE TO RELATED APPLICATIONS

The present application claims priority under 35 U.S.C. §119(e) to U.S. Provisional Application No. 61/233,097 filed on Aug. 11, 2009, the disclosure of which is incorporated by reference herein in its entirety.

### BACKGROUND

1. Field of the Inventions

The present inventions relate to medical devices and, more particularly, to methods and apparatuses for spinal fixation.

2. Description of the Related Art

The human spine is a flexible weight bearing column formed from a plurality of bones called vertebrae. There are thirty-three vertebrae, which can be grouped into one of five regions (cervical, thoracic, lumbar, sacral, and coccygeal). Moving down the spine, there are generally seven cervical vertebrae, twelve thoracic vertebrae, five lumbar vertebrae, five sacral vertebrae, and four coccygeal vertebrae. The vertebrae of the cervical, thoracic, and lumbar regions of the spine are typically separate throughout the life of an individual. In contrast, the vertebra of the sacral and coccygeal regions in an adult are fused to form two bones, the five sacral vertebrae which form the sacrum and the four coccygeal vertebrae which form the coccyx.

In general, each vertebra contains an anterior, solid segment or body and a posterior segment or arch. The arch is generally formed of two pedicles and two laminae, supporting seven processes—four articular, two transverse, and one spinous. There are exceptions to these general characteristics of a vertebra. For example, the first cervical vertebra (atlas vertebra) has neither a body nor spinous process. In addition, the second cervical vertebra (axis vertebra) has an odontoid process, which is a strong, prominent process, shaped like a tooth, rising perpendicularly from the upper surface of the body of the axis vertebra. Further details regarding the construction of the spine may be found in such common references as Gray's Anatomy, Crown Publishers, Inc., 1977, pp. 33-54, which is herein incorporated by reference.

The human vertebrae and associated connective elements are subjected to a variety of diseases and conditions which cause pain and disability. Among these diseases and conditions are spondylosis, spondylolisthesis, vertebral instability, spinal stenosis and degenerated, herniated, or degenerated and herniated intervertebral discs. Additionally, the vertebrae and associated connective elements are subject to injuries, including fractures and torn ligaments and surgical manipulations, including laminectomies.

The pain and disability related to the diseases and conditions often result from the displacement of all or part of a vertebra from the remainder of the vertebral column. Over the past two decades, a variety of methods have been developed to restore the displaced vertebra to their normal position and to fix them within the vertebral column. Spinal fusion is one such method. In spinal fusion, one or more of the vertebra of the spine are united together (“fused”) so that motion no longer occurs between them. The vertebra may be united with various types of fixation systems. These fixation systems may include a variety of longitudinal elements such as rods or plates that span two or more vertebrae and are affixed to the vertebrae by various fixation elements such as wires, staples, and screws (often inserted through the pedicles of the vertebrae). These systems may be affixed to either the posterior or the anterior side of the spine. In other applications, one or more bone screws may be inserted through adjacent vertebrae to provide stabilization.

### SUMMARY

Although spinal fusion is a highly documented and proven form of treatment in many patients, it is contemplated that the rate of bone growth and the quality of the joint formed between fixated bones can be improved. Further, notwithstanding the variety of efforts in the prior art described above, these techniques are associated with a variety of disadvantages. In particular, these techniques typically involve an open surgical procedure, which results higher cost, lengthy in-patient hospital stays and the pain associated with open procedures. Therefore, there remains a need for improved techniques and systems for stabilization and/or fixation of the spine. Preferably, the devices are implantable through a minimally invasive procedure.

Embodiments of the present inventions provide for apparatuses and methods for performing spinal stabilization and/or fixation, for example, such as posterior lumbar stabilization. In particular, it is contemplated that embodiments disclosed herein can achieve better-quality fusion of adjacent vertebrae compared to prior art and apparatuses and methods. In some embodiments, such improvements are provided with apparatuses and methods that stabilize and fixate the spinous processes of adjacent or superior and inferior vertebrae. Additionally, such embodiments can be utilized in conjunction with the placement of other spinal fixation devices, such as bone screws, cages, and the like, which are discussed further herein. In one embodiment, the device for stabilizing and fixating the spinous processes of adjacent or superior and inferior vertebrae and a secondary spinal fixation devices, such as trans-facet or trans pedicle screw can be inserted entirely from a posterior position, substantially posterior position, lateral and/or with the patient lying on their stomach.

In accordance with an embodiment, a method of bone fixation is provided that can comprise: accessing spinous processes of a superior vertebra and an inferior vertebra; forming an aperture in an interspinous ligament between the superior and inferior vertebrae; forming a first notch in the spinous process of the superior vertebra, the first notch facing the spinous process of the inferior vertebra; forming a second notch in the spinous process of the inferior vertebra, the second notch facing the spinous process of the superior vertebra; placing an interspinous process implant such that opposing engagement sections of the implant are fitted against the first and second notches of the respective ones of the superior and inferior vertebrae; and installing a bone fixation device to fix the superior vertebra relative to the inferior vertebra.

In some embodiments, the steps of forming the first notch and forming the second notch can further comprise decorticating the spinous processes of the superior and inferior vertebrae. In some embodiments, there may be only one notch formed, either on the superior vertebra or inferior vertebra. Further, the first and second notches can be formed using a spinous process preparation instrument. The spinous process preparation instrument can comprise a pair of bone cutters. In some embodiments, the preparation instrument can have bone cutters that can form the first and second notch simultaneously. Alternatively, the spinous process preparation instrument can comprise a drill.

The method can further comprise the step of measuring a space between the first notch and the second notch between the spinous processes of the superior vertebra and the inferior vertebra. In embodiments having one notch, the method can comprise the step of measuring the space between the notch and the adjacent spinous process. In this regard, the space can be measured using a distraction tool. Further, the method can further comprise the step of selecting an interspinous process implant based on the measurement of the space between the first notch and the second notch, or in some embodiments between a notch and the adjacent spinous process.

In additional embodiments, the step of placing the interspinous process implant can be performed using an implant delivery tool. For example, the implant delivery tool can comprise a pair of pliers.

Further, the step of installing a bone fixation device can comprise implanting a pair of bone screws into the superior and inferior vertebrae to fix the superior vertebra with respect to the inferior vertebra. Furthermore, the method can further comprise the step of performing a hemi-laminectomy on the inferior vertebra. In some embodiments, the step of installing a bone fixation device can comprise implanting at least one bone screws into superior and inferior vertebrae at one or more spinal levels along the spine to fix the superior vertebra with respect to the inferior vertebra.

Various apparatuses and methods for implanting bone fixation devices and bone graft are provided in U.S. Pat. Nos. 5,893,850, 6,511,481, 6,632,224, 6,648,890, 6,685,706, 6,887,243, 6,890,333, 6,908,465, 6,951,561, 7,070,601, and 7,326,211, and U.S. Patent Application Publication Nos. 2004/0260297, 2004/0127906, 2005/0256525, 2006/0030872, 2006/0122609, 2006/0122610, 2007/0016191, 2008/0097436, 2008/0140207, 2008/0306537, the entirety of the disclosures of which is hereby incorporated by reference herein.

### BRIEF DESCRIPTION OF THE DRAWINGS

The abovementioned and other features of the inventions disclosed herein are described below with reference to the drawings of the preferred embodiments. The illustrated embodiments are intended to illustrate, but not to limit the inventions. The drawings contain the following figures:

FIG. 1 is a side view of an unmodified lumbar spine prior to implantation of a spinal fixation apparatus.

FIG. 2A is a perspective view of the lumbar spine illustrating the use of a punch tool, according to an embodiment.

FIG. 2B is a posterior view of the lumbar spine and punch tool illustrated in FIG. 2A.

FIG. 3A is a side view of an initial step in a procedure for implanting a spinous process implant, in accordance with an embodiment.

FIG. 3B is an enlarged view of bone preparation illustrated in FIG. 3A.

FIG. 4 is a side view of a further step in a procedure for implanting a spinous process implant, in accordance with an embodiment.

FIG. 5A is a side view illustrating forming a notch in a spinous process, in accordance with an embodiment.

FIG. 5B is a side view illustrating forming a notch in another spinous process, in accordance with an embodiment.

FIG. 6 is an enlarged side view of the lumbar spine illustrating inferior and superior spinous processes of inferior and superior vertebrae prepared for receiving the spinous process implant, in accordance with an embodiment.

FIG. 7A is a perspective view of the lumbar spine illustrating the use of a verification tool, according to an embodiment.

FIG. 7B is a posterior view of the lumbar spine and verification tool illustrated in FIG. 7A.

FIG. 8A is a side view of an additional step in the procedure for implanting the spinal process implant, in accordance with an embodiment.

FIG. 8B is an enlarged view of expansion of the inferior and superior spinous processes in FIG. 8A.

FIG. 9 is a perspective view of an interspinous process implant, according to an embodiment.

FIG. 9A is a top view of a modified embodiment of an interspinous process implant.

FIG. 10A is a perspective view of an interspinous process implant, according to another embodiment.

FIG. 10B is a top view of the interspinous process implant illustrated in FIG. 10A.

FIG. 11 is an isometric view of the lumbar spine illustrating initial placement of the interspinous process implant, according to an embodiment.

FIG. 12 is another side view of the lumbar spine illustrating the placement of the interspinous process implant, according to an embodiment.

FIG. 13 is another side view of the lumbar spine illustrating final placement of the interspinous process implant, according to an embodiment.

FIG. 14 is a lateral view of the interspinous process implant after being implanted, according to an embodiment.

FIG. 15 is a posterior view of the interspinous process implant shown in FIG. 14.

FIG. 16 is a posterior view of the interspinous process implant and a pair of facet screws implanted into the inferior and superior vertebrae, according to an embodiment.

FIG. 17 is a lateral view of the interspinous process implant and the pair of facet screws shown in FIG. 16.

FIG. 18 is a posterior view of the inferior and superior vertebrae wherein the interspinous process implant and the pair of facet screws are implanted, and wherein a hemi-laminectomy has been performed, according to an embodiment.

FIG. 19A is a perspective view of a facet screw, according to an embodiment.

FIG. 19B is an enlarged detail view of a proximal end of the facet screw illustrated in FIG. 19A.

FIG. 19C is a top view of a washer illustrated in FIG. 19A.

FIG. 20A is a perspective view of a distraction tool, according to an embodiment.

FIG. 20B is an enlarged detail view of engagement tips of the distraction tool illustrated in FIG. 20A.

FIG. 20C is a top view of the distraction tool in FIG. 20A.

FIG. 21A is a perspective view of a distraction tool, according to another embodiment.

FIG. 21B is an enlarged detail view of engagement tips of the distraction tool illustrated in FIG. 21A.

FIG. 21C is a top view of the distraction tool in FIG. 21A.

FIG. 22A is a perspective view of a distraction tool, according to another embodiment.

FIG. 22B is an enlarged detail view of engagement tips of the distraction tool illustrated in FIG. 22A.

FIG. 22C is a top view of the distraction tool in FIG. 22A.

FIG. 23A is a perspective view of a spinous process preparation instrument, according to an embodiment.

FIG. 23B is a top view of the preparation instrument shown in FIG. 23A.

FIG. 24 is a perspective view of a spinous process preparation instrument, according to another embodiment.

FIG. 25A is a perspective view of a spinous process preparation instrument, according to another embodiment.

FIG. 25B is an enlarged detail view of a cutting end of the spinous process preparation instrument illustrated in FIG. 25A.

FIG. 25C is a top view of the preparation instrument in FIG. 25A.

FIG. 26A is a perspective view of a spinous notch verification tool and handle, according to an embodiment.

FIG. 26B is an enlarged detail view of a verification end of the tool illustrated in FIG. 26A.

FIG. 26C is a top view of the verification tool and handle in FIG. 26A.

FIG. 26D is a front view of the verification tool in FIG. 26A.

FIG. 27A is a perspective view of a verification tool, according to another embodiment.

FIG. 27B is a top view of the verification tool in FIG. 27A.

FIG. 27C is a front view of the verification tool in FIG. 27A.

FIG. 28A is a perspective view of a spinous notch verification tool, according to another embodiment.

FIG. 28B is a top view of the verification tool in FIG. 28A.

FIG. 28C is a front view of the verification tool in FIG. 28A.

FIG. 29A is a perspective view of a spinous notch verification tool, according to another embodiment.

FIG. 29B is a top view of the verification tool in FIG. 29A.

FIG. 29C is a front view of the verification tool in FIG. 29A.

FIG. 30A is a perspective view of an implant delivery tool, in accordance with an embodiment.

FIG. 30B is a top view of the implant delivery shown in FIG. 30A.

FIG. 31A is a perspective view of an implant delivery tool, in accordance with another embodiment.

FIG. 31B is an enlarged detail view of an implant delivery tool illustrated in FIG. 31A.

FIG. 31C is a side view of the implant delivery in FIG. 31A.

### DETAILED DESCRIPTION OF THE PREFERRED EMBODIMENT

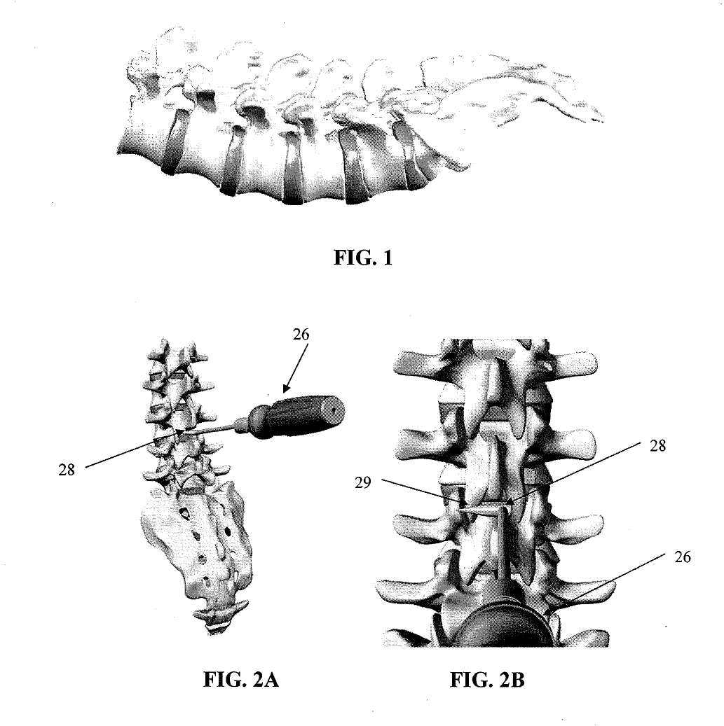

Although the application of the certain embodiments will be initially disclosed in connection with the spinal fixation devices and procedures illustrated in FIGS. 1-27C, the methods and structures disclosed herein are intended for application in any of a wide variety of bones, fixations, and fractures, as will be apparent to those of skill in the art in view of the disclosure herein.

Methods of implanting one or more stabilization devices as part of a spinal stabilization procedure will now be described. Although certain aspects and features of the methods and instruments described herein can be utilized in an open surgical procedure, the disclosed methods and instruments can also be used in the context text of a percutaneous or minimally invasive approach in which the procedure is done through one or more percutaneous small openings. The method steps which follow and those disclosed are intended for use in a trans-tissue approach. However, to simplify the illustrations, the soft tissue adjacent the treatment site have not been illustrated in the drawings.

In an embodiment of use, a patient with a spinal instability is identified. Depending upon the spinal fixation technique, the distal ends of one or more bone fixation devices described herein are advanced into the anterior vertebral body or other suitable portion of one or more vertebrae. As will be explained in more detail below, the stabilization device(s) is typically used to fix the orientation of one vertebra that is unstable, separated or displaced relative to another vertebra, which is not unstable, separated or displaced. However, it should be appreciated that this method may also be applied to three or more vertebrae. In addition, the S-1 portion of the sacrum may be used to stabilize the L5 vertebrae.

The patient is preferably positioned face down on an operating table, placing the spinal column into a normal or flexed position. The target site of a spinal column can then be accessed. Access to the spinal column can be achieved by a mini-open, fully-open procedure, or a percutaneous procedure. In other words, some instruments or devices may be introduced through a mini-open or fully-open procedure to provide access to the target site. In such situations, even small instruments or devices can be inserted through the large passage. However, for smaller instruments or devices, a tissue dilation instrument may be sufficiently large to allow all of the instruments or devices to be passed percutaneously to the target site. In one embodiment, the spinous process spacer described herein and trans-facet screws described herein can be inserted through the same or separate openings. In one arrangement, the spinous process spacer is inserted using a mini-open or fully open procedure while the trans-facet screw can be inserted percutaneously.

An example of a device useful for tissue dilation with a percutaneous procedure is the Teleport Tissue Retractor manufactured by Interventional Spine Inc. The Teleport Tissue Retractor is described in co-pending U.S. Patent Application Publication Nos. 2006/0030872 and 2005/0256525, and PCT Publication No. PCT/US2005/027431 (filed as U.S. patent application Ser. No. 11/659,025 on Jan. 30, 2007). Any of a variety of expandable access sheaths or tissue expanders can be used, such as, for example, a balloon expanded catheter, a series of radially enlarged sheaths inserted over each other, and/or the dilation introducer described in U.S. patent application Ser. No. 11/038,784, filed Jan. 19, 2005 (Publication No. 2005/0256525), the entirety of which is hereby incorporated by reference herein.

In various embodiments disclosed herein, the target site comprises the spinous processes of a superior vertebra and an inferior vertebra. A trocar optionally may be inserted through a tissue tract and advanced towards a first or superior vertebra. In another embodiment, biopsy needle (e.g., Jamshidi™) device can be used. A guidewire may then be advanced through the trocar (or directly through the tissue, for example, in an open surgical procedure) and into the first or superior vertebra. The trajectory and landmarks of the vertebra should be considered in performing this step in order to ensure the proper placement of the treatment site, which will provide placement for the guide wire, fixation device, and/or bone graft material.

After the target site has been accessed, the spinous process is of the superior vertebra and the inferior vertebra can be prepared to receive an interspinous process implant. An exemplary illustration of an unmodified lumbar spine is shown in FIG. 1. The lumbar spine can be prepared in accordance with various embodiments disclosed herein and with reference to the following disclosure and related figures. Additionally, it is contemplated that one of skill in the art may apply the teachings herein in order to carry out various procedures within the scope of the disclosed embodiments.

In some embodiments, a punch tool 26 can be used to form an aperture in an interspinous ligament between superior and inferior vertebrae, as illustrated in FIGS. 2A-2B. The punch tool 26 can have a handle on one end and a piercing end 28. In the illustrated embodiment, the piercing end 28 is a member that is disposed perpendicular to the longitudinal axis of the punch tool 26 and has a sharpened tip 29. The orientation of the piercing end 28 can advantageously create apertures in the interspinous ligament that are lateral to the spine while accessing the vertebrae from the posterior approach. In some embodiments, the piercing end 28 can be disposed parallel to the longitudinal axis of the punch tool 26 to form apertures in the interspinous ligament while accessing the vertebrae from the lateral approach.

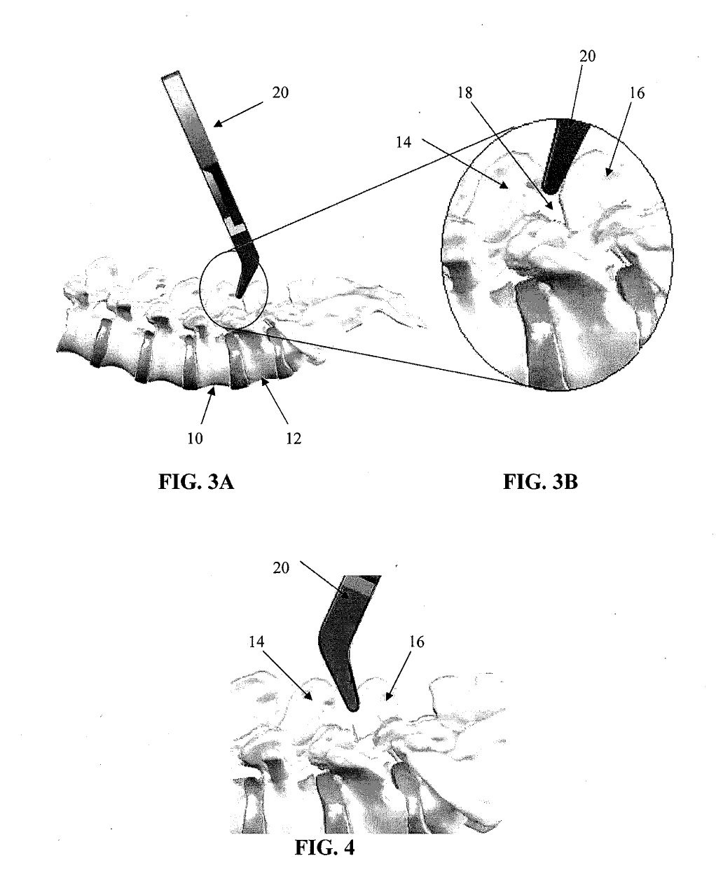

With reference to FIGS. 3A-3B, a superior vertebra 10 and an inferior vertebra 12 are shown. The superior vertebra 10 and the inferior vertebra 12 each comprise a spinous process 14, 16 that define an interspinous process space 18. The spinous processes 14, 16 can be prepared using a bone preparation tool 20 and/or a distraction tool 40. The features and aspects of embodiments of the bone preparation tool 20 and distraction tool 40 will be discussed further below.

Continuing, the bone preparation tool 20 and/or distraction tool 40 can be inserted into the interspinous process space 18 in order to contact opposing portions of the spinous process 14, 16. The preparation tool 20 and/or distraction tool 40 can be used to modify the shape and/or surface structure of the spinous processes 14, 16. For example, the preparation tool 20 and/or distraction tool 40 can remove material from the spinous processes 14, 16. The material can be removed to create a desired structure in the spinous processes 14, 16. The material can be removed, for example, by roughening, rasping, cutting, or notching the spinous processes 14, 16. Further, the amount of material removed may be minimal. That is, in some embodiments, the shape of the spinous process is not modified but is instead merely roughened. This process of removing material from the spinous process can create bleeding bone, which aids in promoting fusion. In this regard, it is contemplated that the preparation tool 20 and/or distraction tool 40 can be used to make mating features in the spinous processes 14, 16. Such mating features can comprise structures such as notches and the like, which will be described further below. Additionally, the preparation tool 20 and/or distraction tool 40 can be used to decorticate one or more edges or surfaces of at least one of the spinous processes 14, 16. In this manner, the preparation tool 20 and/or distraction tool 40 can be used to create bleeding bone conducive to fusion. Accordingly, the spinous processes 14, 16 can be modified in a variety of ways using the preparation tool 20 and/or distraction tool 40 in order to prepare the interspinous process space 18 for receiving an implant and encouraging bone fusion.

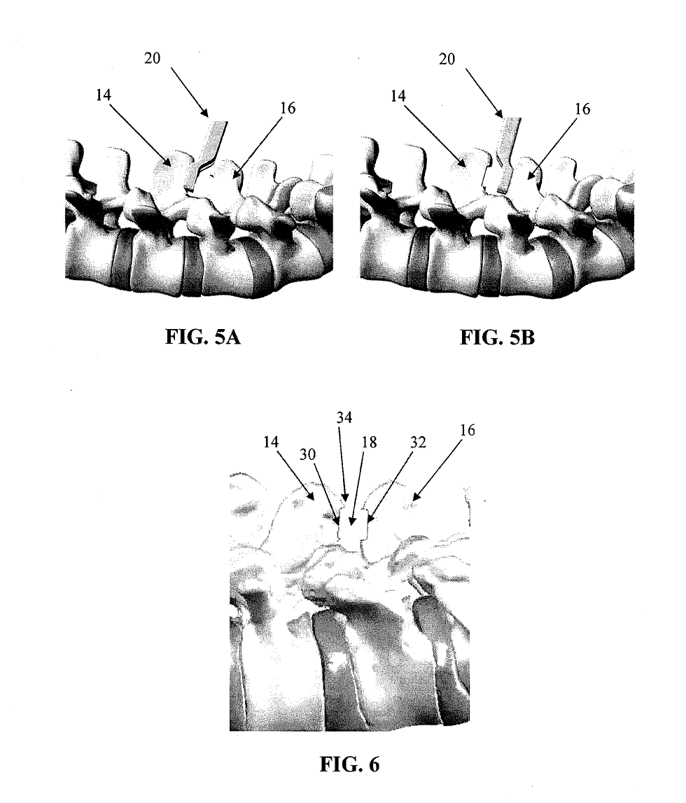

In some embodiments illustrated in FIGS. 4-6, the tool 20 can be used to create notches 30, 32 in the respective ones of the spinous processes 14, 16. The notches 30, 32 can be formed on opposing portions of the spinous processes 14, 16 in the interspinous process space 18. In some embodiments, a notches 30, 32 can be formed in an at least two step process, wherein a first notch is formed on a spinous process and then the tool 20 is repositioned to make a second notch on the opposing spinous process, as illustrated in FIGS. 5A-5B. In some embodiments, the tool 20 can comprise a pair of bone cutters, wherein both notches can be formed at the same time. In other embodiments, the tool 20 can comprise a drill. In some embodiments, the notches 30, 32 can be at least partially formed by the distraction tool 40, which can be configured to rasp the spinous processes. As such, a tool, such as bone cutters, rasping devices or a drill can be used to accomplish a notching or process modification procedure in order to form the desired structure and create bleeding bone.

As discussed further below, the notches 30, 32 can act as a locator stops that prevent migration of an interspinous process implant into spinal dura. Accordingly, the notches 30, 32 can be formed to define a shape or structure that can be complementary to that of a corresponding interspinous process implant. The preparation of the processes 14, 16 in such embodiments can provide significant advantages and superior results for maintaining a desired positional relationship of the implant with the spinous processes. Moreover, the overall effectiveness of the fusion process can be enhanced.

In some embodiments, at least one of the protrusions 34 adjacent the notches 30, 32 can reduced, angled, or rounded for easier insertion of the interspinous process implant during the implant procedure. In some embodiments, at least one of the notches 30, 32 can be extended to the outer edge of the spinous process 14, 16 such that a protrusion 34 is removed. To maintain the implant in position after implantation, a temporary or permanent method can be used, such as adhesives, fasteners, clips, etc.

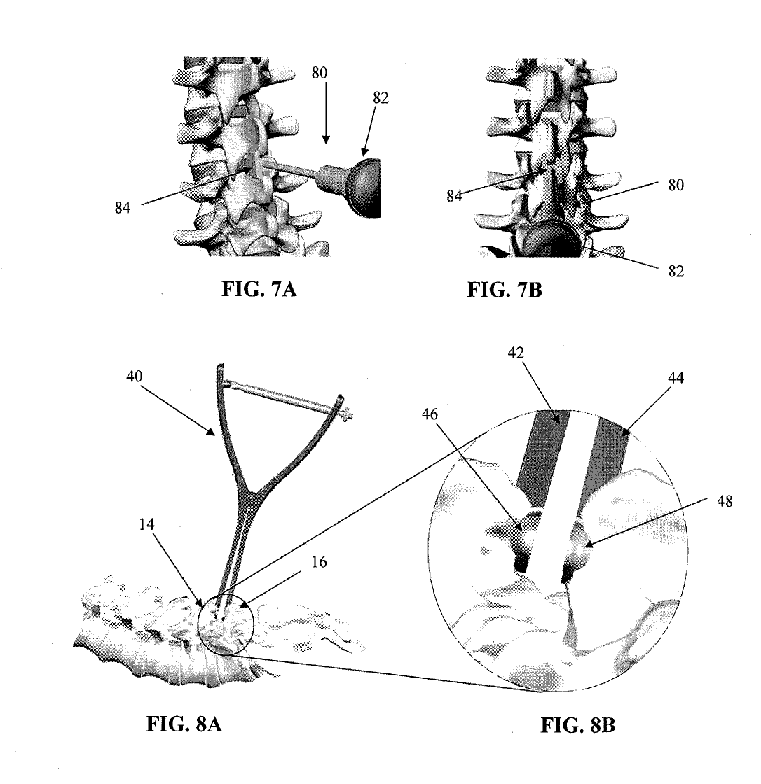

As illustrated in FIGS. 7A-7B, a verification tool 80 can be used to check that the thickness of the spinous processes is compatible with the width of the implant 50, as described further below. The verification tool 80 can be inserted from a posterior approach to fit onto articular processes, with or without notches. In some embodiments, the verification tool can be configured to be inserted from a lateral approach.

Some of the embodiments of the implantation method can be modified to comprise the step of measuring the interspinous process space 18. As illustrated in FIGS. 8A-8B, a distraction tool 40 can be inserted into the interspinous process space 18 in order to measure the size or distance between the surfaces (and in some embodiments, the notches 30, 32) of the spinous processes 14, 16. In some embodiments, the distraction tool 40 can also distract the space between the spinous processes to relieve pressure on the disc and nerves, as discussed further below. The measurement obtained using the distraction tool 40 can be used to select an implant having the appropriate shape and size. Some embodiments of distraction tools 40, 140, 240 will be described further below. Some embodiments of distraction tools 140, 240 can be configured to rasp or cut the notches in the articular processes.

As shown in FIG. 8B, the distraction tool 40 can comprise a pair of separating arms 42, 44 having engagement tips 46, 48. The engagement tips 46, 48 can be configured to engage with the respective notches of the spinous processes 14, 16. In performing the measurement function, the distraction tool 40 can be used to manipulate the interspinous process space 18 to determine the optimal size for an implant. As such, the distraction tool 40 can be configured to engage the spinous processes 14, 16 in a manner similar to that in which the implant will engage the spinous processes 14, 16.

In some embodiments, the distraction tool 40 can be used to separate or distract the spinous processes 14, 16. The distraction tool 40 can be positioned so that the engagement tips 46, 48 engage with the spinous processes 14, 16, as described above, and the separating arms 42, 44 can be operated to separate the spinous processes 14, 16. In some embodiments, the measurement device on the distraction tool 40 can be used to measure the separation of the spinous processes 14, 16 to the desired distance in order to accept an interspinous process implant 50.

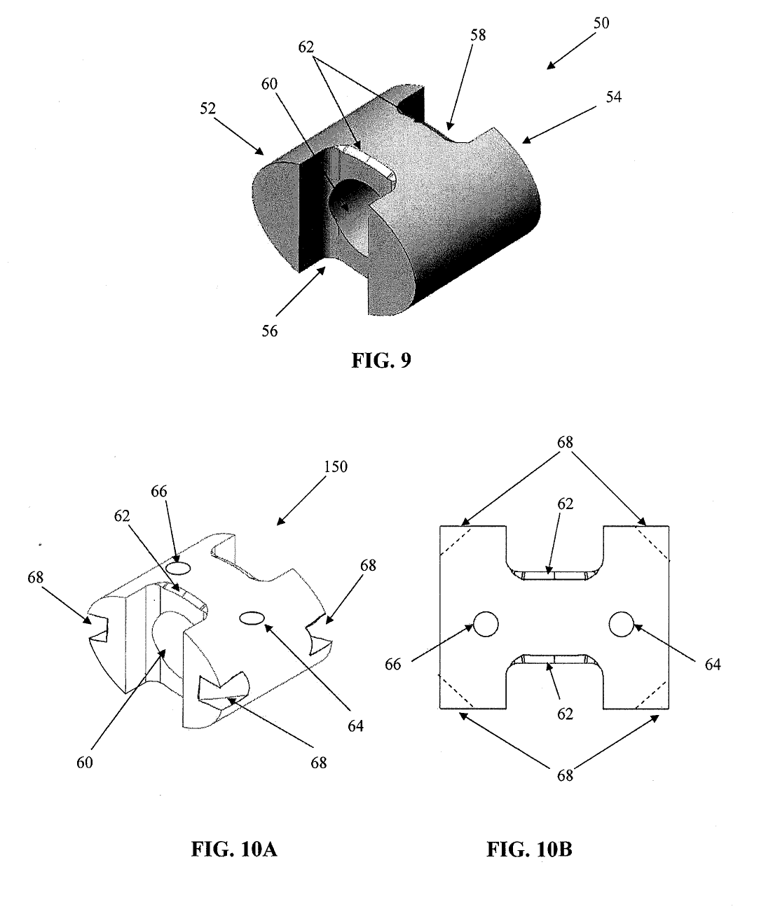

An embodiment of an interspinous process spacer or implant 50 is shown in FIG. 9. As illustrated, the interspinous process spacer or implant 50 can be a device having a ovular cross-section and comprising first and second ends 52, 54. The implant 50 can be an allograft implant (e.g., machined from cortical ring allograft derived from human cadaveric tissue) or made of any suitable biocompatible material. The first and second ends 52, 54 can be configured to engage opposing spinous processes of a superior vertebra and an inferior vertebra. In the illustrated embodiment, the first and second ends 52, 54 can comprise an indentation or groove 56, 58 configured to receive at least a portion of the respective spinous process. In this manner, the spacer or implant 50 can be securely seated onto the interspinous process space against the spinous processes. In other words, the indentation or groove 56, 58 of the first and second ends 52, 54 can serve to limit lateral movement of the spacer or implant 50 relative to the spinous processes. Moreover, in some embodiments in which the spinous processes are modified to comprise a notch or similar structure, the spacer or implant 50 can be constrained from anterior-posterior movement. In some embodiments, the implant 50 can include chamfers 62 on at least the inner edges of the indentation or groove 56, 58, as illustrated in FIG. 9. The chamfers 62 can help to provide clearance when the implant 50 is inserted past the protrusions 34 on the spinous processes 14, 16. In some embodiments, the chamfers can help by decreasing the insertion forces needed to insert the implant 50. The chamfer 62 can have any angle to help provide clearance for the implant 50 to be positioned between spinous processes 14, 16. In some embodiments, the angle between the chamfer 62 and the lateral plane of the implant 50 can be at least about 5 degrees and/or less than or equal to about 85 degrees. In some embodiments, the angle can be at least about 30 degrees and/or less than or equal to about 60 degrees. Embodiments of the process and apparatus provided herein can enable a surgeon to place an interspinous process implant in a manner that ensures reliable placement of the implant and reliable stabilization of the superior and inferior vertebrae.

In some embodiments, the spacer or implant 50 can also comprise one or more apertures or through holes 60. The aperture 60 can extend along a vertical or superior-inferior axis of the implant 50. However, other apertures can be provided that extend in a lateral or anterior posterior direction. It is contemplated that the apertures can enhance the osseointegration of the bone with the implant, and more particularly, bone growth between the inferior and superior vertebrae. In some embodiments, the aperture 60 can be at least partially filled with demineralized bone matrix (DBM) or other bone graft material to enhance osseointegration. The aperture 60 can provide a longitudinal graft port to provide a pathway for osteoinductive graft material promoting osteointegration between adjacent spinous processes.

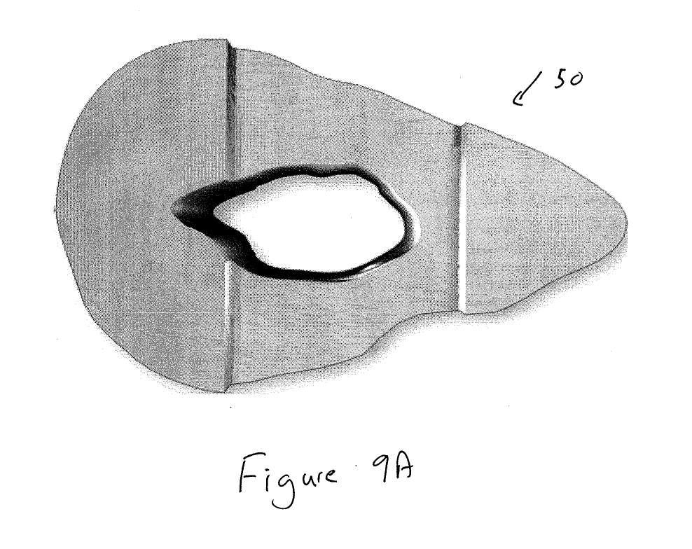

With reference to FIGS. 10A-10B, the implant 150 can include tool engagement apertures 64, 66 that extend laterally through the implant 150 that are configured to accept an implant delivery tool. In some embodiments, the apertures 64, 66 can have a circular cross-sectional shape that can accept pins on the implant delivery tool. In some embodiments, the apertures 64, 66 can have other cross-sectional shapes, such as ovular, square, rectangle, hexagonal, or other shape that can accept a complementary shaped extension on the implant delivery tool. The apertures 64, 66 can be internally threaded so that they can be attached to the implant delivery tool with a fastener. In some embodiments, instead of or in addition to the apertures 64, 66 the spacer can have notches on the sides that can accept arms of the implant delivery tool. The apertures 64, 66 or notches can allow the implant delivery tool to hold the implant 150 while reducing the size of the implant delivery tool, which advantageously allows the implant 150 to be implanted in confined spaces.

In some embodiments, the corners of the implant 150 can have corner notches 68, as illustrated in the embodiment of FIGS. 10A-10B. The corner notches 68 can be configured to accept a holding or locking band that can be wrapped around the spinous processed 14, 16, and the implant 150 to secure the implant 150 in position after implantation. In addition to the illustrated embodiments, the implant 50 can include an overall taper from front to back as shown in FIG. 9A. For example, the general diameter of the implant 50 can be larger at the front end as compared to the back end so as to correspond to the anatomy of the spinous process. In other embodiments, the taper can be reversed or modified. In embodiments in which the implant 50 is made from allograft, the implant may have an irregular shape corresponding to the bone from which the implant 50 is harvested from.

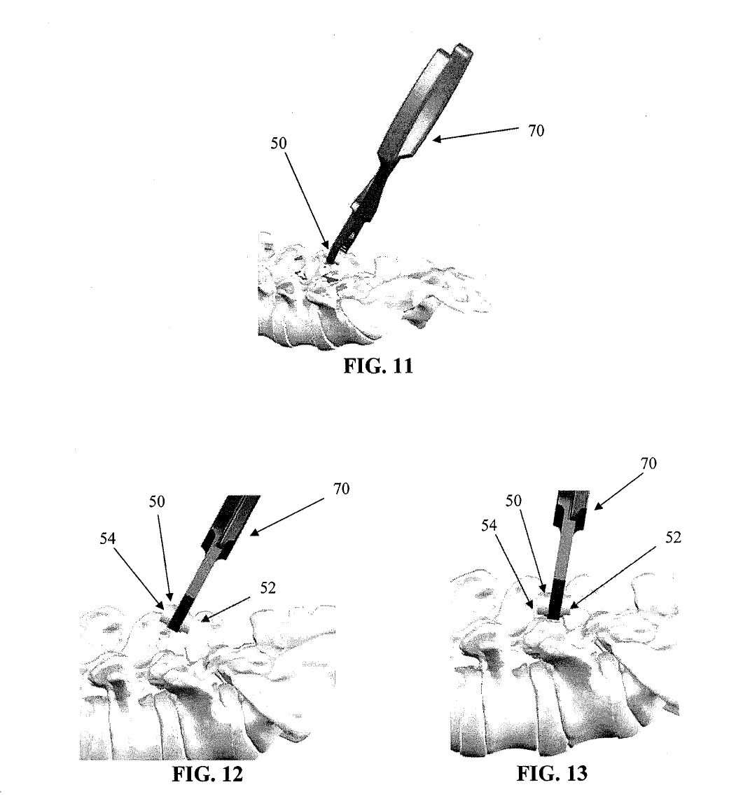

Referring now to FIGS. 11-13, embodiments of the process can further comprise inserting an implant between the spinous processes. For example, a properly sized and specially selected implant can be inserted between the prepared spinous processes, in accordance with the disclosure and teachings above. The placement of the implant can be performed using an implant delivery tool 70.

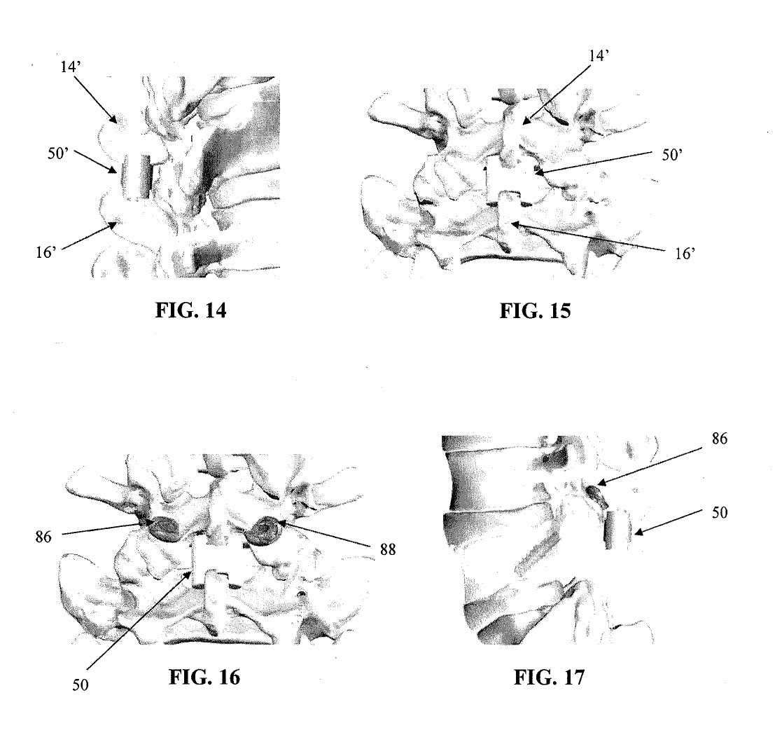

As illustrated in FIGS. 11-13, the implant delivery tool 70 can be used to place a first end 52 and then a second end 54 of the implant such that the implant engages the spinous processes in a desirable manner. FIGS. 14-15 illustrate an implant 50′ that has been properly placed between interspinous processes 14′, 16′ in a desirable manner.

After the interspinous process implant has been placed, it is also contemplated that one or more additional fixation devices can be used to stabilize the superior vertebra with respect to the inferior vertebra. Possible bone fixation devices and methods of use are shown and described in further detail in U.S. Pat. No. 6,951,561 and U.S. Patent Application Publication Nos. 2004/0127906, 2007/0118132, and 2007/0123868, the entireties of the disclosures of which are hereby incorporated by reference herein.

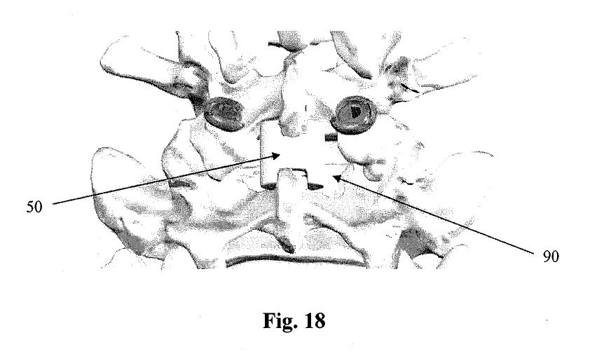

For example, as illustrated in FIGS. 16-17, one or more bone screws 86, 88 (e.g., transfacet-pedicular screws) can be used to fix the superior vertebra with respect to the inferior vertebra. In these embodiments, the bone screw 86, 88 can be inserted through a facet of a superior vertebral body into a pedicle and/or facet of an inferior vertebral body. In certain embodiments, a ratcheting screw can be used, as described below and disclosed in U.S. Pat. No. 6,951,561 issued Oct. 4, 2005 entitled SPINAL STABILIZATION DEVICE, which is incorporated by reference in its entirety herein. In some embodiments, a standard screw having a threaded shaft and a fixed head can be used. In some embodiments, a threaded shaft can be inserted into the facets and secured with a nut on the proximal end. In some embodiments, a lag screw having two threaded portions can be used, wherein one threaded portion has a greater thread pitch than the other threaded portion, such that the two facets through which the lag screw is implanted are joined together as the lag screw is turned. Further, in a modified embodiment, as shown in FIG. 18, a laminectomy or hemi-laminectomy 90 can optionally be performed on the inferior and/or superior vertebrae. A system using the bone screws 86, 88 and implant 50 can advantageously provide spinal stabilization without the need for implanting cages in the intervertebral disk space. In addition, in certain embodiments, the bone screws 86, 88 and the implant 50 can advantageously be inserted into the patient while the patient is lying face down without having to shift the position of the patient. In such embodiments, the implant 50 can be used to support the posterior fixation devices (e.g., bone screws 86, 88) by providing a third column of support thereby sharing the axial load with the posterior fixation devices. Such embodiments can be a less invasive alternative to fixation systems that require anterior or interbody fusion procedures. In other embodiments, the implant 50 can be used in combination with pedicle screws and rod systems.

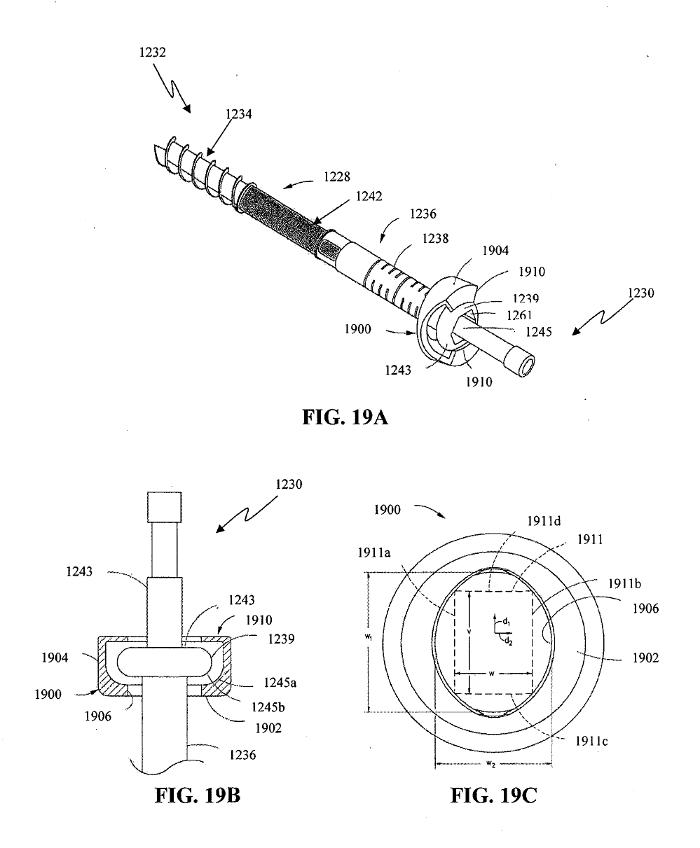

Referring to FIGS. 19A-19C, the ratcheting screw 1000 can comprise a pin body 1228 extending between a proximal end 1230 and a distal end 1232. The length, diameter and construction materials of the body 1228 can be varied, depending upon the intended clinical application. In one embodiment, the body 1228 comprises titanium. However, other metals or bioabsorbable or nonabsorbable polymeric materials may be utilized, depending upon the dimensions and desired structural integrity of the finished ratcheting screw 1000.

The distal end 1232 of the body 1228 is provided with a cancellous bone anchor or distal anchor 1234. In general, the cancellous bone anchor 1234 is adapted to be rotationally inserted into the facets, to retain the ratcheting screw 1000 within the facets.

The proximal end 1230 of the body 1228 is provided with a proximal anchor 1236. The proximal anchor 1236 is axially distally moveable along the body 1228, to permit compression of the facets. Complimentary locking structures such as threads or ratchet like structures between the proximal anchor 1236 and the body 1228 resist proximal movement of the anchor 1236 with respect to the body 1228 under normal use conditions. The proximal anchor 1236 can be axially advanced along the body 1228 either with or without rotation, depending upon the complementary locking structures as will be apparent from the disclosure herein.

In the illustrated embodiment, proximal anchor 1236 comprises a housing 1238 such as a tubular body, for coaxial movement along the body 1228. The housing 1238 is provided with one or more surface structures such as radially inwardly projecting teeth or flanges, for cooperating with complementary surface structures 1242 on the body 1228. The surface structures and complementary surface structures 1242 permit distal axial travel of the proximal anchor 1236 with respect to the body 1228, but resist proximal travel of the proximal anchor 1236 with respect to the body 1228. Any of a variety of complementary surface structures which permit one way ratchet like movement may be utilized, such as a plurality of annular rings or helical threads, ramped ratchet structures and the like for cooperating with an opposing ramped structure or pawl.

Retention structures 1242 are spaced axially apart along the body 1228, between a proximal limit and a distal limit. The axial distance between proximal limit and distal limit is related to the desired axial range of travel of the proximal anchor 1236, and thus the range of functional sizes of the ratcheting screw 1000. In one embodiment of the ratcheting screw 1000, the retention structure 1242 comprise a plurality of threads, adapted to cooperate with the retention structures on the proximal anchor 1236, which may be a complementary plurality of threads. In this embodiment, the proximal anchor 1236 may be distally advanced along the body 1228 by rotation of the proximal anchor 1236 with respect to the body 1228. Proximal anchor 1236 may be advantageously removed from the body 1228 by reverse rotation, such as to permit removal of the body 1228 from the patient.

Tensioning and release of the proximal anchor 1236 may be accomplished in a variety of ways, depending upon the intended installation and removal technique. For example, a simple threaded relationship between the proximal anchor 1236 and body 1228 enables the proximal anchor 1236 to be rotationally tightened as well as removed. However, depending upon the axial length of the threaded portion on the pin 1228, an undesirably large amount of time may be required to rotate the proximal anchor 1236 into place. For this purpose, the locking structures on the proximal anchor 1236 may be adapted to elastically deform or otherwise permit the proximal anchor 1236 to be distally advanced along the body 1228 without rotation, during the tensioning step. The proximal anchor 1236 may be removed by rotation as has been discussed. In addition, any of a variety of quick release and quick engagement structures may be utilized. For example, the threads or other retention structures surrounding the body 1228 may be interrupted by two or more opposing flats. Two or more corresponding flats are provided on the interior of the housing 1238. By proper rotational alignment of the housing 1238 with respect to the body 1228, the housing 1238 may be easily distally advanced along the body 1228 and then locked to the body 1228 such as by a 90° or other partial rotation of the housing 1238 with respect to the body 1228. Other rapid release and rapid engagement structures may also be devised, and still accomplish the advantages of the present embodiments.

With continued reference to FIGS. 19A-19C, an embodiment of a flange or washer 1900 is illustrated. The washer 1900 is configured to interact with the head 1239 of the proximal anchor 1236. The washer 1900 includes a base 1902 and a side wall 1904. The base 1902 and side wall 1904 define a curved, semi-spherical or radiused surface 1245a that interacts with the corresponding curved, semi-spherical or radiused surface 1245b of the head 1239. The surface 1245a surrounds an aperture 1906 formed in the base 1902. As described above, this arrangement allows the housing 1238 and/or body 1228 to extend through and pivot with respect to the washer 1900.

With particular reference to FIG. 19C, in the illustrated embodiment, the aperture 1906 is elongated with respect to a first direction d1 as compared a second direction d2, which is generally perpendicular to the first direction d1. In this manner, the width w1 of the aperture in the first direction is greater than the width w2 of the aperture in the second direction. In this manner, the aperture 1906 provides a channel 1911 with a width w between the sides 1911a, 1911b defined with respect to the second direction d2 that is preferably greater than the maximum width of the tubular housing 1238 but smaller than the width of the head 1908 such that the proximal anchor 1236 can not be pulled through the aperture 1906. The height h of the channel is defined between the sides 1911c, 1911d in the second direction. As such, the elongated aperture 1906 permits greater angular movement in a plane containing the first direction d1 as portions of the proximal anchor 1236 are allowed rotate into the elongated portions of the aperture 1906. The aperture 1906 may be elliptical or formed into other shapes, such as, for example, a rectangle or a combination of straight and curved sides.

The washer 1900 optionally includes a portion that is configured so that the proximal end 1243 of the anchor 1236 is retained, preferably permanently retained, within the washer 1900. In the illustrated embodiment, the side walls 1904 are provided with lips 1910. The lips 1910 extend inwardly from the side walls 1904 towards the aperture 1906 and interact with the proximal end 1243 of the head 1239 so that the proximal anchor 1236 is retained within the washer 1900. Preferably, the washer 1900 is toleranced to allow the proximal anchor 1236 to freely rotate with respect to the washer 1900. In this manner, the washer 1900 and the proximal anchor 1236 can move together for convenient transport.

As described above, when the body 1228, the proximal anchor 1236 and the washer 1900 are deployed into a patient, the washer 1900 can inhibit distal movement of the body 1228 while permitting at least limited rotation between the body 1228 and the washer 1900. As such, the illustrated arrangement allows for rotational and angular movement of the washer 1900 with respect to the body 1228 to accommodate variable anatomical angles of the bone surface. This embodiment is particularly advantageous for spinal fixation and, in particular, trans-laminar, trans-facet and trans-facet-pedicle applications. In such applications, the washer 1900 may seat directly against the outer surface of a vertebra. Because the outer surface of the vertebra is typically non-planar and/or the angle of insertion is not perpendicular to the outer surface of the vertebra, a fixed flange may contact only a portion of the outer surface of the vertebra. This may cause the vertebra to crack due to high stress concentrations. In contrast, the angularly adjustable washer 1900 can rotate with respect to the body and thereby the bone contacting surface may be positioned more closely to the outer surface. More bone contacting surface is thereby utilized and the stress is spread out over a larger area. In addition, the washer, which has a larger diameter than the body 1228, or proximal anchor described herein, effectively increases the shaft to head diameter of the fixation device, thereby increasing the size of the loading surface and reducing stress concentrations. Additionally, the washer 1900 can be self aligning with the outer surface of the vertebra, which may be curved or non-planer. The washer 1900 can slide along the surface of the vertebra and freely rotate about the body 1228 until the washer 1900 rests snugly against the surface of the vertebra for an increased contact area between the bone and the washer 1900. As such, the washer 1900 can be conveniently aligned with a curved surface of the vertebra.

In some embodiments, one or more fixation devices may be inserted into the vertebrae with bilateral symmetry such that such two vertebrae are coupled together with two or more fixation devices on a left side of the spine being connected using one or more rods and/or plates to two or more fixation devices on a right side of the spine. In certain of these embodiments, the distal anchor of these fixation devices may be inserted through the pedicle and/or the facet of the vertebrae. In other embodiments, the fixation devices will be utilized to secure adjacent vertebral bodies in combination with another fusion procedure or implant, such as the interspinous process implant disclosed herein or a spinal cage, plate or other device for fusing adjacent vertebral bodies. Thus, the fixation devices may operate in conjunction with a cage or other implant to provide three-point stability across a disc space, to assist in resisting mobility between two vertebral bodies. In other embodiments, the fixation device may simply be advanced through a portion of a first vertebra and into a second, preferably adjacent, vertebra. In certain of these embodiments, the fixation device may extend through the facet of the first vertebra and the distal anchor may be inserted through the facet or pedicle of the second vertebra.

In addition to the above, it is contemplated that embodiments of the method can be modified to include other preparation steps, such as rasping intervertebral joint space or using bone graft material, as disclosed in Applicant's copending patent application Ser. No. 12/821,980, filed Jun. 23, 2010, the entirety of the disclosure of which is incorporated herein by reference, in order to enhance the stabilization results.

Further, it is noted that the devices and procedures discussed herein can be used to address spinal stenosis. In this regard, distraction of the vertebrae can help relieve pressure of the nerve roots, and the implant 50 can be used to maintain the distraction. In other words, in some embodiments, the implant 50 can hold the vertebrae apart at a desired distance while the fixation devices can be used to stabilize the orientation of the vertebrae. The fixation devices across the facet/pedicles can then be used to secure the vertebrae and promote fusion.

The access site may be closed and dressed in accordance with conventional wound closure techniques and the steps described above may be repeated on the other side of the vertebrae for substantial bilateral symmetry. The bone stabilization devices may be used alone or in combination with other surgical procedures such as a hemi-laminectomy, laminectomy, discectomy, artificial disc replacement, and/or other applications for relieving pain and/or providing stability.

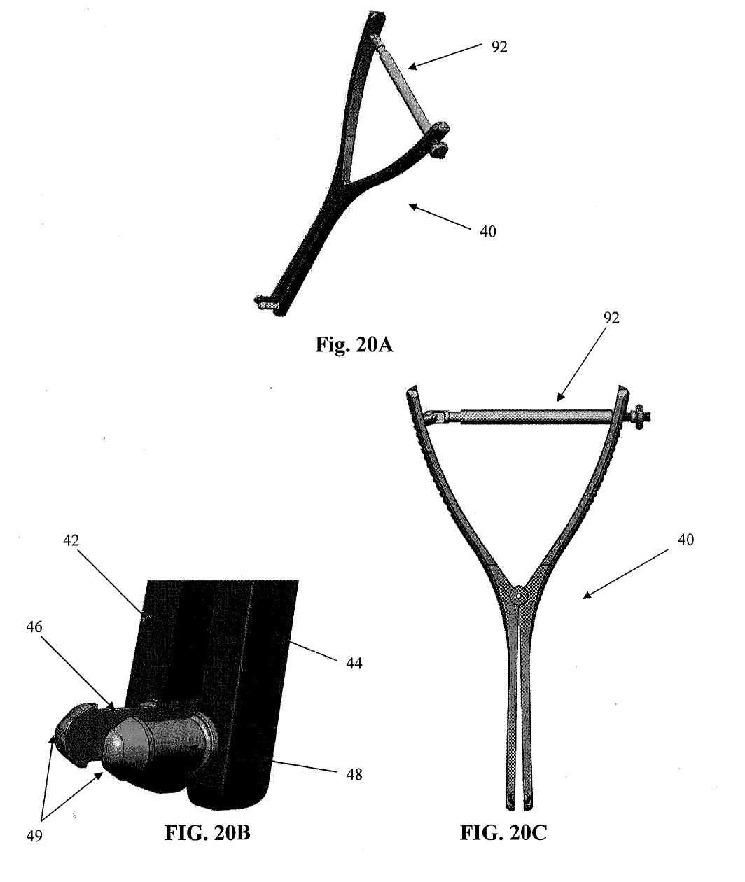

FIGS. 20A-20C illustrate views of the distraction tool or measurement instrument 40 discussed above. The distraction tool 40 can comprise a pair of separating arms 42, 44 having engagement tips 46, 48. The engagement tips 46, 48 can be configured to engage with the spinous processes. The distraction tool or measurement instrument 40 can be used to distract the spinous process is to a desired distance or spacing, as described above. The tool 40 can also display the implant size to accurately assess the correct implant to use. For this purpose, the tool 40 can comprise a measurement component 92. Furthermore, the grooves and features of the engagement tips 46, 48 can allow the tool 40 to properly mate with the spinous processes without slipping. For example, as illustrated in FIG. 20B, each engagement tip 46, 48 can be shaped as a half cylinder with a bulbous tip 49, wherein the two engagement tips 46, 48 make up a complete cylindrical extension with a bulbous tip 49. The spinous process is placed on the engagement tips 46, 48 and the bulbous tips 49 help secure the distraction tool 40 without slipping off the spinous processes. In some embodiments, the distraction tool 40 can be made of a rigid material that is sufficiently strong to separate adjacent spinous processes. For example, the distraction tool 40 can be made of material such as metals (e.g. aluminum, titanium), plastics (e.g. HDPE), polymers (e.g., PEEK), or composites.

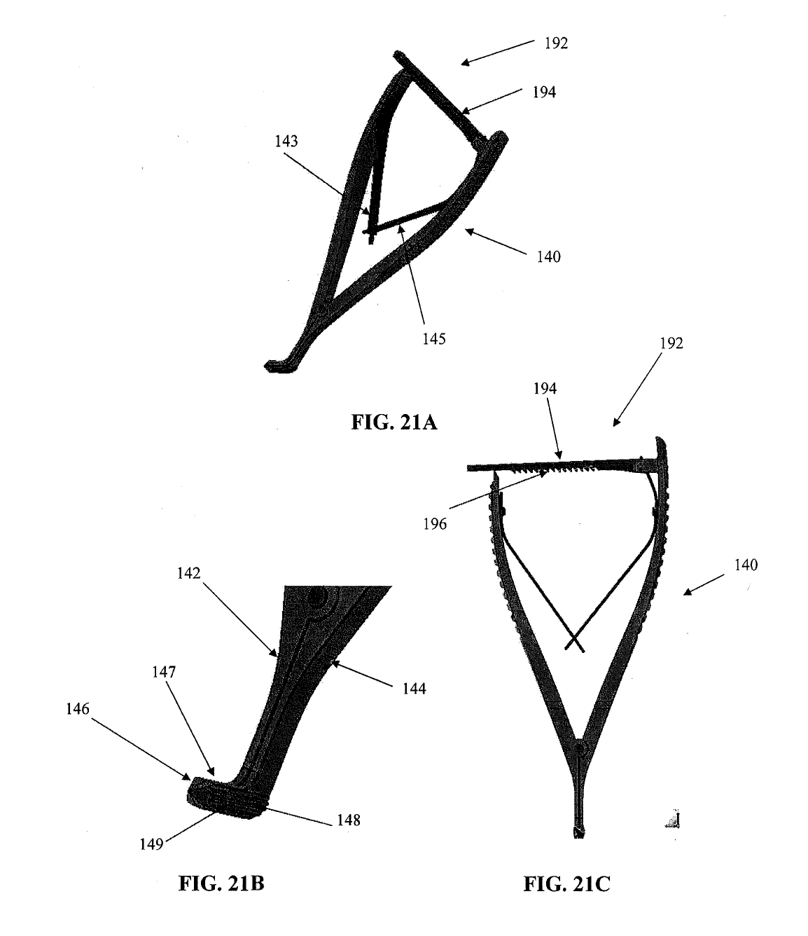

FIGS. 21A-21C illustrate another embodiment of the distraction tool 140. The distraction tool 140 includes a pair of separating arms 142, 144 having engagement tips 146, 148. The distraction tool 140 can have at least one biasing member 143, 145 that biases the distraction tool 140 in the closed configuration. In some embodiments, the biasing members 145, 147 can be made of a resilient material, such as spring steel, plastics or composites. As illustrated in FIG. 21B, the engagement tips 146, 148 can have pointed ends. In some embodiments, the pointed ends can be used to pierce ligaments and access the interspinous process space 18. In some embodiments, the engagement tips 146, 148 can have textured surfaces 147, 149, such as the grooves in the illustrated embodiment. The textured surfaces 147, 149 can be used for filing or rasping the surfaces of the spinous processes 14, 16 to form notches. In some embodiments, as described above, the rasping of the spinous processes can create bleeding bone and aid in promoting fusion.

With continued reference to FIGS. 21A and 21C, the tool 140 can have a measurement component 192 that displays the implant size to accurately assess the correct implant to use. In the illustrated embodiment, the measurement component 192 is a notched member 194 that is connected to one handle and couples with an end of the other handle as the handles are squeezed together. The measurement notches 196 are angled so that the end of the handle can move in the closing direction, but is obstructed by the measurement notches 196 from opening. In some embodiments, the notched member 194 can be biased toward the engagement tips by the biasing member 145. The side of the notched member 194 can have markings indicating the correct implant size to use according to the distance that the handles are squeezed together. In other embodiments, the measurement component can be any other device that displays the implant size as the distraction tool 140 is operated.

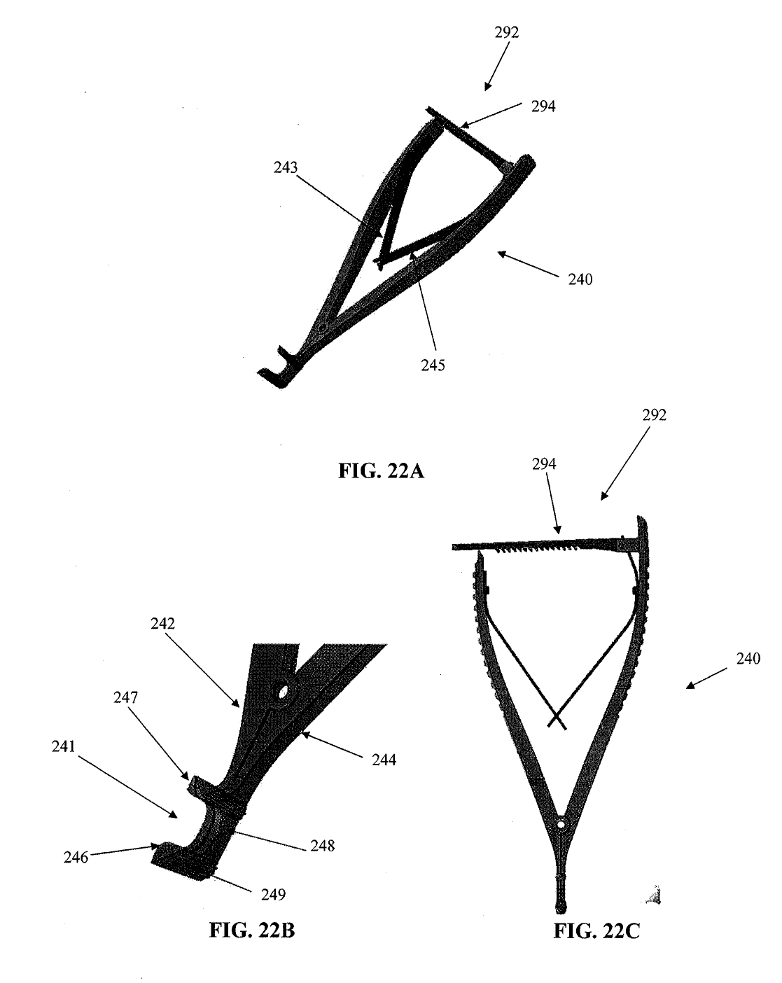

FIGS. 22A-22C illustrate another embodiment of the distraction tool 240. The distraction tool 240 includes a pair of separating arms 242, 244 having engagement tips 246, 248. The distraction tool 240 can have at least one biasing member 243, 245 and measurement component 292, as described above in other embodiments. As illustrated in FIG. 22B, the engagement tips 246, 248 can be generally C-shaped and have an implant space 241 for holding the implant or maintaining the space in which the interspinous implant 50 can be placed. In some embodiments, the distraction tool 240 can be used to separate the spinous processes 14, 16 and hold the implant for implantation into the interspinous process space 18. The ends of the C-shaped engagement tips 246, 248 can be pointed as described above, for example to pierce ligaments and access the interspinous process space 18. In some embodiments, the engagement tips 246, 248 can have textured surfaces 247, 249, such as the grooves in the illustrated embodiment. The textured surfaces 247, 249 can be used for roughening or rasping the surfaces of the spinous processes 14, 16, which as described above can create bleeding bone and aid in promoting fusion.

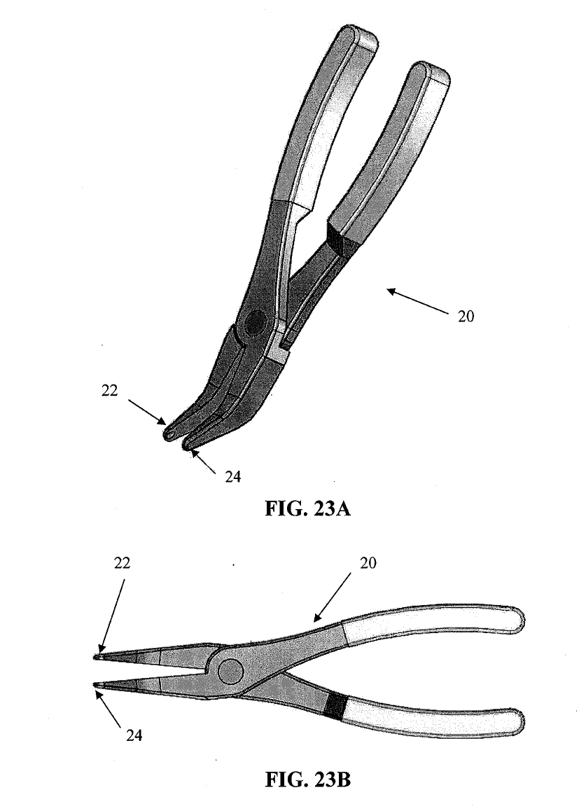

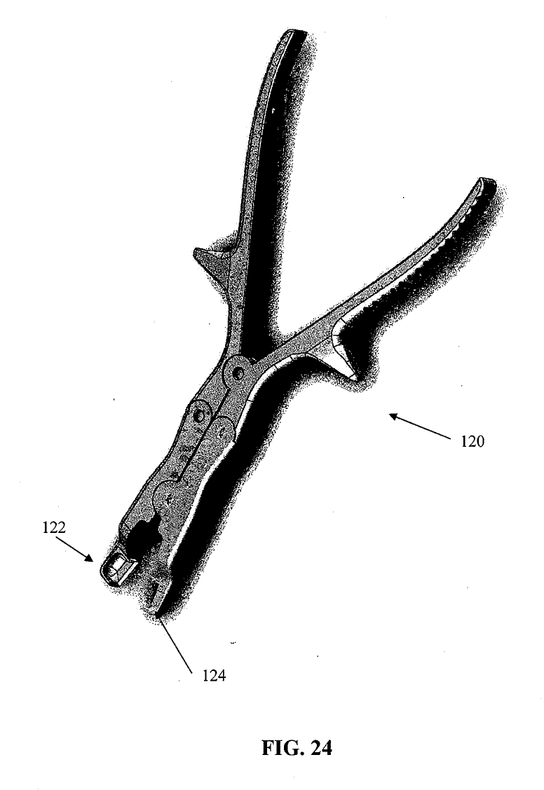

Further, FIGS. 23A-23B illustrate views of the bone preparation tool or spinous process preparation instrument 20 described above. The instrument 20 can be used to cut away edges of cortical bone of the spinous processes. Additionally, the instrument 20 can be used to create a notch in the spinous process feature for the implant to sit in. In some embodiments, the tool 20 can be oriented to make the first notch and then the tool 20 can be repositioned to make the second notch. In some embodiments, tips 122, 124 of the instrument 120 can be uniquely configured with a bone cutting geometry in order to efficiently and accurately cut the spinous processes. As illustrated in FIG. 24, the bone preparation tool 120 can be configured to make a preset shaped cut in a direction.

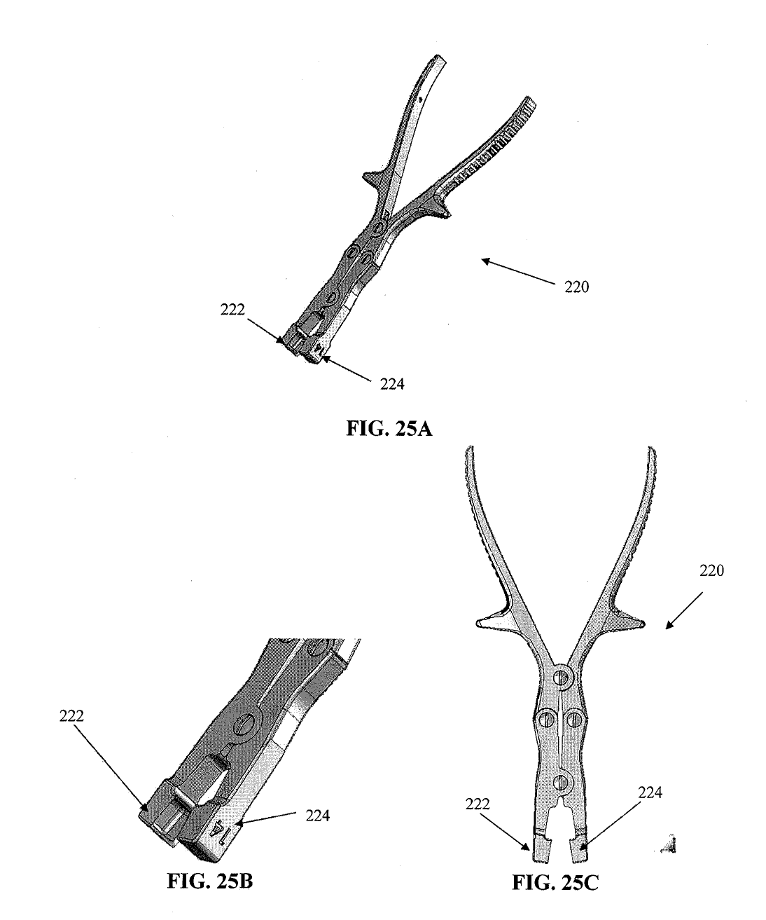

FIGS. 25A-25C illustrate another embodiment of a bone preparation tool 220. The bone preparation tool 220 can have cutting tips 222, 224 that are configured to form the notches 30, 32 by cutting away edges of cortical bone of the spinous processes 14, 16. The cutting tips 222, 224 can be configured to form the proper sized and shaped notches 30, 32. In some embodiments, both notches 30, 32 can be formed at the same time by the double sided cutting tips 222, 224. Forming both notches 30, 32 at the same time can advantageously produce cuts that are substantially parallel and opposed to provide good union with the implant. Furthermore, the preparation tool 220 can be positioned so that the notches are equal in depth in each spinous process, or in some embodiments, the notches can be positioned so that one notch is deeper or more shallow than the opposing notch in the spinous process. In other embodiments, one notch can be formed by the double sided cutting tips 222, 224 and then the other notch can be formed. In these situations, the double sided cutting tips 222, 224 advantageously allow the user to make both notch cuts without having to turn the bone preparation tool 220 around, such as in the embodiment described above. In some embodiments, the cutting tips can have multiple sizes and/or shapes so that the same bone preparation tool can be used to make different sized and/or shaped notches.

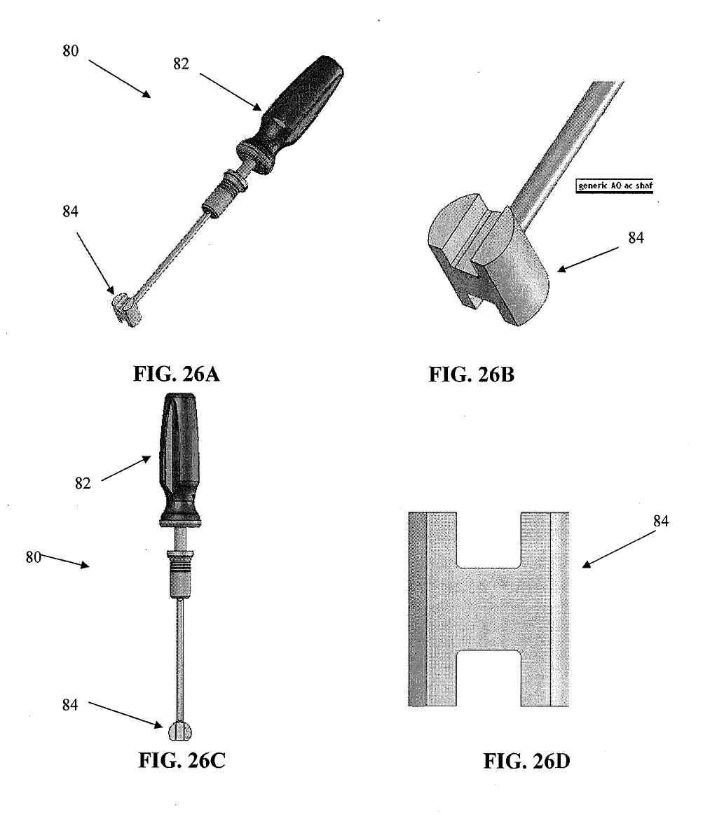

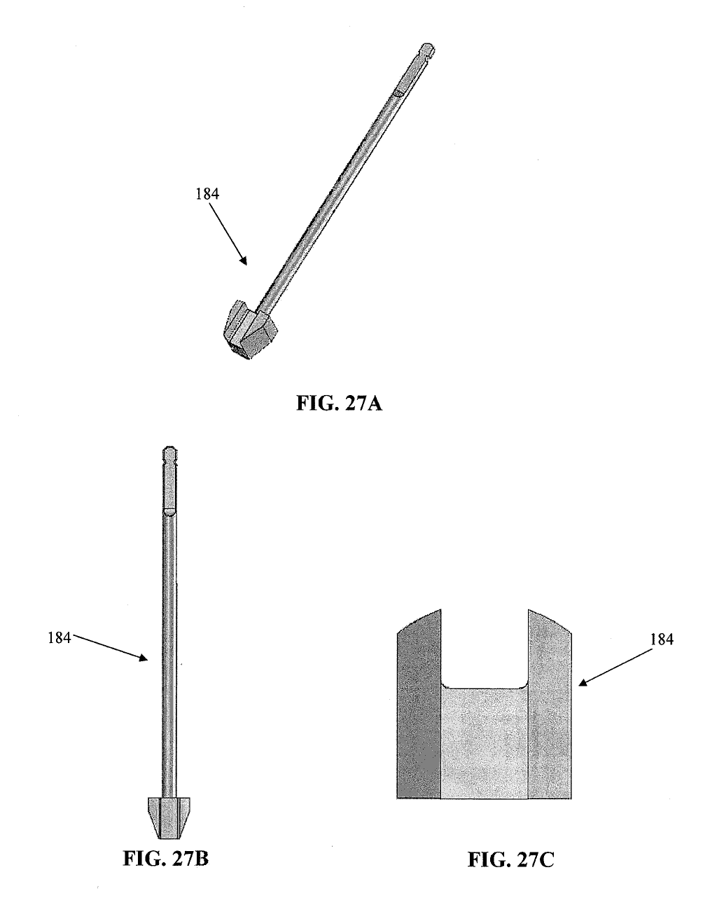

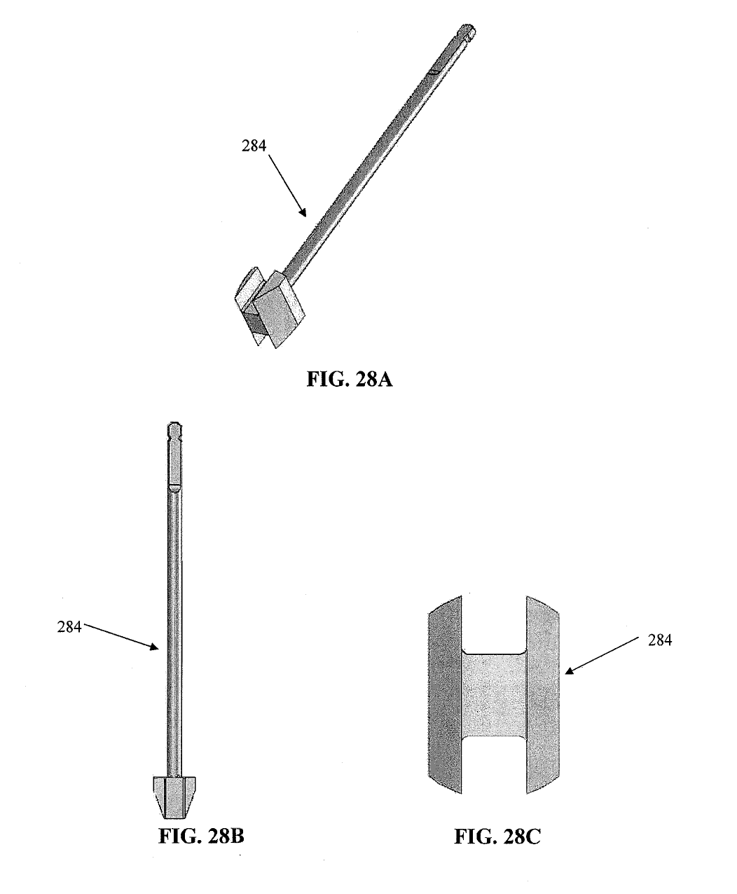

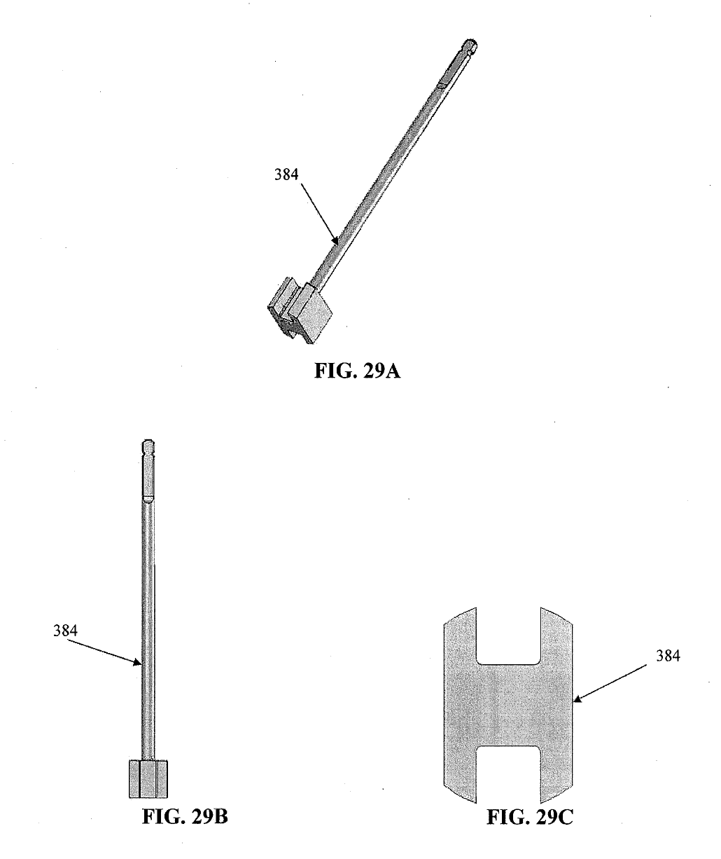

FIGS. 26A-26D illustrates a verification tool 80 having a handle 82 and a verification tip 84. The verification tip 84 can be releasably attached to the handle 82 through any coupling mechanism known in the art, so that the verification tip 84 can be interchangeable. For example, as illustrated in FIG. 27A, the verification tip 84 can have a flat portion that provides anti-rotation and coupling with the handle 82. In some embodiments, the handle 82 can be integrated with the verification tip 84 wherein the verification tip 84 is not detachable from the handle 82. The verification tip 84 can be shaped and sized similar to the implant 50 that is to be implanted into the interspinous process space 18. The verification tip 84 can be used to check the length of the interspinous process space 18 and the size of the notches 30, 32 before the actual implant 50 is inserted. For example, the verification tip 84 can be used to check that the thickness of the spinous processes is compatible with the width of the implant 50. It can be useful to use the verification tool 80 because it is easier to insert and remove the verification tool 80 compared to the actual implant 50. For example, the implant 50 can be made of a fragile material, such as allograft, which can get damaged from repeated insertion and removal when checking the interspinous process space 18. The verification tool 80 can advantageously be made of a strong material, such as plastics, polymers (e.g., PEEK) or metals, but can also be yielding to some extent to reduce the risk of damage the spinous processes 14, 16. FIGS. 27A-29C illustrates alternate embodiments of the verification tool. In some embodiments, the front edge of the verification tool can be angled or sharpened, which can be used in preparing the width of the spinous processes to accept the implant 50 and/or create bleeding bone to encourage bony fusion.

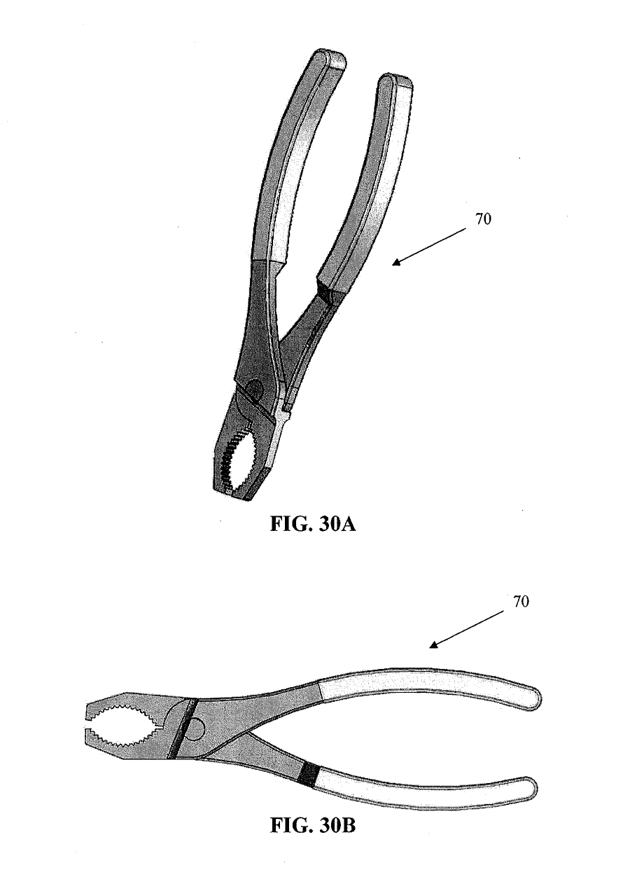

FIGS. 30A-30B illustrate views of the implant delivery tool 70 described above. The implant delivery tool 70 can be configured with geometry that enables the tool 70 to hold implant of varying geometries. As illustrated, and the tool 70 can comprise a rounded interior structure with a plurality of grooves to securely grip the implant. Further, the tool 70 can be configured to allow the user to introduce and rotate the implant into position between the spinous processes.

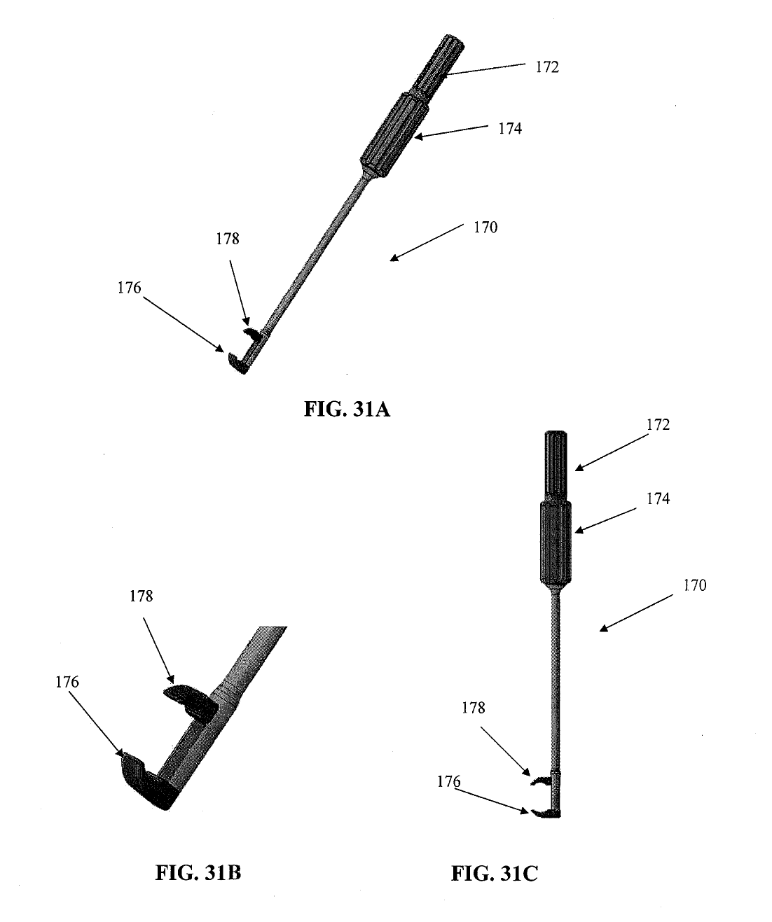

FIGS. 31A-31C illustrate an alternate embodiment of an implant delivery tool 170. The implant delivery tool 170 has a first handle portion 172 and a second handle portion 174. At the other end of the implant tool 170 are the first grip 176 and second grip 178. The first and second grips 176, 178 are configured with geometry to hold implants of varying geometries. The second grip 178 can be moveable along the longitudinal length of the implant tool 170. In some embodiments, the movement of the second grip 178 can be actuated by manipulation of the handles. For example, the second handle portion 174 can be coupled to a rod that is connected to the second grip 178. Rotation of the second handle portion 174 about the longitudinal axis can translate to lateral movement of the rod and the connected second grip 178, thus gripping the implant. In some embodiments, the delivery tool can have an angled portion between the handles and grips, wherein the delivery tool can be inserted into the patient from the posterior direction. In some embodiment, the delivery tool 170 can have a generally straight portion between the handles and grips, as illustrated in the figures, wherein the delivery tool 170 can be inserted into the patient from a lateral direction. In some embodiments, the delivery tool 170 can be used when the spine is accessed through the posterior approach.

The specific dimensions of any of the embodiment disclosed herein can be readily varied depending upon the intended application, as will be apparent to those of skill in the art in view of the disclosure herein. Moreover, although the present inventions have been described in terms of certain preferred embodiments, other embodiments of the inventions including variations in the number of parts, dimensions, configuration and materials will be apparent to those of skill in the art in view of the disclosure herein. In addition, all features discussed in connection with any one embodiment herein can be readily adapted for use in other embodiments herein to form various combinations and sub-combinations. The use of different terms or reference numerals for similar features in different embodiments does not imply differences other than those which may be expressly set forth. Accordingly, the present inventions are intended to be described solely by reference to the appended claims, and not limited to the preferred embodiments disclosed herein.

What is claimed is:

1. A method of bone fixation comprising:
accessing spinous processes of a superior vertebra and an inferior vertebra;
forming a first notch in the spinous process of the superior vertebra, the first notch facing the spinous process of the inferior vertebra;
forming a second notch in the spinous process of the inferior vertebra, the second notch facing the spinous process of the superior vertebra;
placing an interspinous process implant such that opposing engagement sections of the implant are fitted against the first and second notches of the respective ones of the superior and inferior vertebrae; and
installing a bone fixation device to fix the superior vertebra relative to the inferior vertebra.

2. The method of claim 1, further comprising the step of forming an aperture in an interspinous ligament between the superior vertebra and the inferior vertebra after accessing the spinous processes.

3. The method of claim 1, wherein the steps of forming the first notch and forming the second notch further comprise decorticating the spinous processes of the superior and inferior vertebrae.

4. The method of claim 1, wherein the first and second notches are formed using a spinous process preparation instrument.

5. The method of claim 1, wherein the spinous process preparation instrument comprises a pair of bone cutters.

6. The method of claim 1, wherein the spinous process preparation instrument comprises a drill.

7. The method of claim 1, further comprising the step of measuring a space between the first notch and the second notch between the spinous processes of the superior vertebra and the inferior vertebra.

8. The method of claim 7, wherein the space is measured using a distraction tool.

9. The method of claim 7, further comprising the step of selecting an interspinous process implant based on the measurement of the space between the first notch and the second notch.

10. The method of claim 1, wherein the step of placing the interspinous process implant is performed using an implant delivery tool.

11. The method of claim 10, wherein the implant delivery tool comprises a pair of pliers.

12. The method of claim 1, wherein the step of installing a bone fixation device comprises implanting a pair of bone screws into the superior and inferior vertebrae to fix the superior vertebra with respect to the inferior vertebra.

13. The method of claim 1, further comprising the step of performing a hemi-laminectomy on the inferior vertebra.

14. A method of bone fixation comprising:
accessing spinous processes of a superior vertebra and an inferior vertebra;
placing an implant such that opposing engagement sections of the implant are fitted against the spinous processes of the respective ones of the superior and inferior vertebrae.

15. The method of claim 14, further comprising the step of forming a notch in at least one of the spinous processes before placing the implant.

16. The method of claim 14, further comprising the step of installing a posterior fixation device to fix the superior vertebra relative to the inferior vertebra.

17. The method of claim 16, wherein the step of installing a posterior fixation device comprising inserting a pair of screws with bilateral symmetry between the superior and inferior vertebrae.
